(19) United States
(12) Patent Application Publication
(10) Pub. No.: US 2007/0042008 A1
(43) Pub. Date: Feb. 22, 2007

(54) Compositions containing phosphatidylcholine and essential fatty acids

(75) Inventors: Edward Kane, Millville, NJ (US); Patricia Kane, Millville, NJ (US)

Correspondence Address: LAW OFFICES OF KHALILIAN SIRA, LLC, 9100 PERSIMMON TREE ROAD, POTOMAC, MD, 20854, US

(73) Assignee: BodyBio, Inc., Millville, NJ (US)

(21) Appl. No.: 11/206,925

(22) Filed: Aug. 18, 2005

(51) Int. Cl. A61K 31/737; A61K 31/22; A61K 31/685; A61K 9/00

(52) U.S. Cl. 424/400; 514/78; 514/54; 514/547

(57) ABSTRACT

The invention discloses pharmaneutical compositions comprising a phospholipid formulation that is fluidized with an oil composition containing a pre-determined ratio of omega 6 and omega 3 fatty acids. In particular the invention discloses phosphatidylcholine compositions that are fluidized with a pre-determined ratio of linoleic acid and alpha linolenic acid.

### Field of the Invention

This invention relates to compositions containing phospholipids and essential fatty acids. In particular, the invention relates to compositions of phosphatidylcholine fluidized with a balanced ratio of essential fatty acid composition.

### BACKGROUND OF THE INVENTION

Phospholipidic compositions have long been known and used in various ways in nutrition, cosmetics, and pharmaceutical industries. The majority of products containing phospholipids have to be further fluidized by the addition of oil.

Composition containing phospholipids or essential fatty acids and their use in treating central nervous system (CNS) related diseases or disorders have been discovered before. For example, U.S. Pat. No. 6,852,870 discloses the use of omega 3 fatty acids, and omega 3 phoshatidylcholine to treat patients with major depression.

U.S. Patent Application No 20010033872 discloses compositions containing phospholipids and fatty acids for the treatment or prevention of mental disturbances such as depressive states and for regulating the level of neurotransmitters and thereby improving the function of the central nervous system and cognitive function in humans.

Various formulations of compositions containing phospholipids are also disclosed. For example, U.S. Pat. No. 6,278,004 discloses stabilized phospholipid compositions containing at least one phospholipid mixed with a stabilizer such as phytic acid or a salt of phytic acid.

U.S. Pat. No. 5,015,483 is directed to liposome compositions prepared by dissolving a lipophilic material in a phospholipid followed by the addition of water or an aqueous solution and mixing by sonicating to producing a liposome having the lipophilic material encapsulated in the lipid bilayer. The preferred lipophilic materials include omega 3 fatty acids.

Phosphatidylcholine (PC) is the predominant phospholipid of all cell membranes and of the circulating blood lipoproteins. PC is the main lipid constituent of the lipoprotein particles circulating in the blood and the preferred precursor for certain phospholipids and other biologically important molecules. PC also provides antioxidant protection in vivo. In animal and human studies, PC protected against a variety of chemical toxins and pharmaceutical adverse effects.

Chemically, PC is a glycerophospholipid that is built on glycerol (CH2OH—CHOH—CH2OH) and substituted at all three carbons. Carbons 1 and 2 are substituted by fatty acids and carbon 3 by phosphorylcholine. Simplistically, the PC molecule consists of a head-group (phosphorylcholine), a middle piece (glycerol), and two tails (the fatty acids, which vary). Variations in the fatty acids in the tails account for the great variety of PC molecular species in human tissues. In vivo, PC is produced via two major pathways. In the predominant pathway, two fatty acids (acyl “tails”) are added to glycerol phosphate (the “middle piece”), to generate phosphatidic acid (PA) that is converted to diacylglycerol, after which phosphocholine (the “head-group”) is added on from CDP-choline. The second, minor pathway is phosphatidylethanolamine (PE) methylation, in which the phospholipid PE has three methyl groups added to its ethanolamine head-group, thereby converting it into PC.

Taken orally PC is very well absorbed, up to 90% per 24 hrs when take with meals. PC enters the blood gradually and its levels peak over 8-12 hours. During the digestive process, the position 2 fatty acid becomes detached (de-acylation) in the majority of the PC molecules. The resulting lyso-PC readily enters intestinal lining cells, and is subsequently re-acylated at this position. The position 2 fatty acid contributes to membrane fluidity (along with position 1), but is preferentially available for eicosanoid generation and signal transduction. The omega 6/omega 3 balance of the PC fatty acids is subject to adjustment via dietary fatty acid intake. Choline is most likely an essential nutrient for humans, and dietary choline is ingested predominantly as PC. Greater than 98 percent of blood and tissue choline is sequestered in PC that serves as a “slow-release” blood choline source.

Methyl group (—CH3) availability is crucial for protein and nucleic acid synthesis and regulation, phase-two hepatic detoxification, and numerous other biochemical processes involving methyl donation. Methyl deficiency induced by restricted choline intake is linked to liver steatosis in humans, and to increased cancer risk in many mammals. PC is an excellent source of methyl groups, supplying up to three per PC molecule, and is the main structural support of cell membranes, the dynamic molecular sheets on which most life processes occur. Comprising 40 percent of total membrane phospholipids, PC's presence is important for homeostatic regulation of membrane fluidity. PC molecules of the outermost cell membrane deliver fatty acids on demand for prostaglandin/eicosanoid cellular messenger functions, and support signal transduction from the cell's exterior to its interior.

Long-chain fatty acids serve as precursors of phospholipids in the body and may have independent neurochemical effects as well. Exogenously administered fatty acids are combined within the cell with phosphate moieties donated by ATP molecules to form phospholipids. Omega 3 fatty acids have been linked to the etiology, pathophysiology, and treatment of unipolar major depression. Several lines of evidence support a role for the omega 3 fatty acids in major depression.

Unlike omega 3 fatty acids, omega 6 fatty acids are ubiquitous in developed countries. Omega 6 fatty acids are derived from seed and vegetable oils that have increased in the Western diet through the incorporation of these oils by the food industry and at the recommendation of the American Heart Association and others (Report of the Dietary Guidelines Advisory Committee on the Dietary Guidelines for Americans. In: Dietary Guidelines for Americans; Washington, D.C. US Dept of Health and Human Services; 2000). There is indirect evidence that the optimal dietary ratio of omega-6 to omega-3 fatty acids should be close to 1:1, and under these optimal conditions, the omega-6 fatty acid competes with the omega-3 fatty acid to achieve balanced immune and inflammatory function.

Omega 6 and omega 3 fatty acids have to be in balance in order to provide the body with maximum immune protection. The imbalance of these fatty acids increases the risk for intense and uncontrollable inflammatory responses. For example, an imbalance towards the omega-6 eicosanoids causes white blood cells to release potent immune activating cytokines, which can adversely affect health if chronically or abnormally activated. Likewise, omega 3 fatty acids can dramatically check this process at several levels, including direct inhibition of the eicosanoid producing enzyme cyclooxygenase-2. See, for example, Obata T, et. al., EICOSAPENTAENOIC ACID INHIBITS PROSTAGLANDIN D2 GENERATION BY INHIBITING CYCLO-OXYGENASE-2 IN CULTURED HUMAN NAST CELLS. Clin. Exp. Allergy; 1999, 2:1129-1135).

There is a need in the art, therefore, for a composition that combines multiple mechanisms of action to increase effectiveness of each of the components of the composition and at the same time contains ingredients having a wide margin of safety, no side effects and immense health benefits. The invention described herein solves the long felt need by providing novel compositions utilizing specific formulations of phospholipids fluidized with a balanced ratio of essential oil composition that restore a healthy balance of essential nutrients paramount to maintain or restore the health of the individual.

### SUMMARY OF THE INVENTION

The invention as disclosed herein provides phamaneutical compositions comprising a phospholipid formulation that is fluidized with an essential oil composition containing a pre-determined ratio of omega 6 and omega 3 fatty acids and a suitable carrier. The phospholipid used in the composition of the invention comprises phosphatidylserine, phosphatidylcholine, phosphatidylethanolamine, phosphatidylglycerol, phosphatidylinositol, phosphatidic acid, cardiolipin, sphingomyelin, ceramide, gangliosides, cerebrosides, or a combination thereof.

In a preferred embodiment, the phospholipid comprises phosphotidylcholine. The phosphotidylcholine has one or two acyl groups. In one embodiment, the acyl groups have derived from myristic acid, palmitic acid, stearic acid, oleic acid, linoleic acid, linolenic acid, arachidic acid, arachidonic acid, or a combination thereof.

In one embodiment, the oil composition comprises a pre-determined ratio of linoleic acid and alpha linolenic acid, preferably in the ratio of about 4:1, respectively.

In another embodiment, the oil composition is about 20% and the phosphotidylcholine is about 80% of total weight of the pharmaneutical composition.

In another embodiment, the omega 6 and omega 3 fatty acids are derived from a blend of organic fatty acids that are cold pressed.

In another aspect, the invention provides kits comprising a phospholipid formulation, an oil composition containing a pre-determined ratio of omega 3 and omega 6 fatty acids, instructions for mixing the phospholipid formulation to obtain a fluidized phospholipid composition, and instructions to use the fluidized phospholipid composition. The phospholipid formulation is preferably a phosphatidylcholine in a liquid or dry formulation.

In one embodiment, the kit additionally contains color, flavor, preservatives, antibacterial agents, or a combination thereof.

Other preferred embodiments of the invention will be apparent to one of ordinary skill in the art in light of what is known in the art, in light of the following description of the invention, and in light of the claims.

### DETAILED DESCRIPTION OF THE INVENTION

The invention as described herein provides Pharmaneutical compositions containing one or more phospholipids fluidized with a pre-determined amount of an oil composition containing a balanced ratio of omega 6 and omega 3 essential oils.

As used herein, a “pharmaneutical composition” includes any composition in which at least 50% of its compounds, compositions and/or constituents have been derived from natural sources and/or are used in their natural form, as opposed to being chemically, or synthetically produced.

As used herein, an “effective amount” of a composition is an amount sufficient to achieve a desired biological effect, in this case at least one of prevention, amelioration or treatment of a specific disease or disorder that responds positively to the administration of phosphatidylcholine. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

As used herein, a “carrier” refers to a diluent, adjuvant, excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is a preferred carrier when the pharmanuetical composition is administered intravenously. Saline solutions and aqueous dextrose and glycerol solutions can also be employed as liquid carriers, particularly for injectable solutions.

As used herein “phospholipids” also refer to as phosphatides and phospholipins, are lipids which contain a phosphoric acid or derivative thereof. Glycerophospholipids, have been referred to as phosphatides and phosphoglycerides, are any glycerophosphoric acid or derivative thereof with one or two acyl, alkenyl or alkyl groups attached to a glycerol residue. These materials may be prepared synthetically or may be derived from natural sources. Natural sources that produce phospholipids are commonly seeds as well as animal products such as eggs.

As used herein, “Omega 3 fatty acids” refer to long chain polyunsaturated molecules beginning with a methyl group and ending with a carboxyl group. Omega-3 fatty acids contain a double bond in the third position from the methyl group. Two common, long chain omega-3 fatty acids are eicosapentanoic acid (20 carbons in length) and docosahexanoic acid (22 carbons in length). These are both found in fish oils As used herein, “omega 3 phosphatidylcholine” refers to a triacylglycerol in which the gamma carbon of glycerol is esterified to phosphocholine, and at least one of the other carbons of glycerol is esterified to an omega 3 fatty acid.

As used herein, “triacylglycerol” are compounds in which the carboxyl groups of fatty acids are esterified to the hydroxyls of all three carbons found in glycerol are referred to as triacylglycerols or triglycerides. Triacylglycerols in which the terminal carbon of glycerol (the “gamma carbon”) is esterified to phosphocholine are called phosphatidylcholine. The next carbon in the glycerol is referred to herein as the “beta carbon” and the following carbon is referred to as the “alpha carbon”.

As used herein, “choline” refers to hydroxyethyl trimethyl ammonium hydroxide that is considered to be a vitamin of the B complex and is derivable from many foods. Unless otherwise indicated, the term “choline” as used herein, refers not only to the isolated choline molecule (i.e. free choline) but also to any biologically compatible salt of choline (e.g., choline bitartrate).

The phospholipids are a class of lipid molecules of particular importance in the function of the mammalian cell. They make up 75% of cell membranes, and therefore play an essential role in interactions between cells, including the communications that pass between neurons in the brain. Phospholipids are responsible for maintaining cell membrane fluidity. In other words, phospholipids allow cell receptors to be mobile over the surface of the cell, thereby enhancing cell-to-cell communication. This is important in all tissues, but especially so in the central nervous system (CNS). Phospholipids increase the effectiveness of existing levels of neurotransmitters, by ensuring that a maximum number of neurotransmitter molecules are able to interface with a receptor molecule.

There are two general classes of phospholipids, namely, phosphoglycerides and sphingolipids. The two groups of phospholipids are interrelated in both structure and function. Both are amphipathic, that is, they have a hydrophilic head composed of a phosphate group and a long hydrophobic tail composed of one or more fatty acid chains. Attached to the phosphate head are various moieties that characterize the different phospholipids. It is the amphipathic nature of phospholipids that enables them to function in the cell membrane. Examples of phosphoglycerides include phosphatidylserine, phosphatidylcholine, distearoylphosphatidylcholine (DSPC), dilinleyolphosphotidylcholine, lephosphatidic acid, phosphatidylethanolamine, phosphatidylglycerol, phosphatidylinositol, and cardiolipin, or a combination thereof among others.

Phosphatidylserine is the precursor of phosphatidylcholine, which is the single most abundant phospholipid in mammalian cell membranes. Examples of sphingolipids that are essential to the structure and function of the CNS include sphingomyelin, ceramide, gangliosides, and cerebrosides. The preferred phospholipids in the compositions of the present invention are phosphatidylserine, phosphatidylcholine, sphingomyelin, or a combination thereof.

Additionally, the phospholipid component can comprise one or more lysophospholipids. Lysophospholipids are single chain phospholipids. Examples of lysophospholipids include lysophosphatidylcholines, such as monopalmitoylphosphatidylcholine (MPPC), lysophosphatidylglycerols, lysophosphatidylethanolamines, lysophosphatidylinositols, lysophosphatidylserines, and lysophosphatidic acid. Mixtures of different types of phospholipids and/or lysophospholipids can also be used.

The most preferred phospholipids used in the composition of the invention are phsosphatidylcholine. Phosphatidylcholine is a class of phospholipids called “essential phospholipids.” Although the structure of phosphatidylcholine is somewhat similar to other phospholipids, there are some differences that have profound biological and physiological effects. Humans synthesize choline in small amounts not sufficient for the bodily requirements. Choline and the compounds derived from it serve many vital functions. Most of the choline in the human body is located in phosphatidylcholine.

Sphingomyelin (or ceramide phosphorylcholine) consists of a ceramide with a phosphorylcholine moiety attached to position 1. It is thus the sphingolipid analogue of phosphotidylcholine. It is a ubiquitous component of animal cell membranes, where it is by far the most abundant sphingolipid. Indeed, it can comprise as much as 50% of the lipids in certain tissues, though it is usually less abundant than phosphatidylcholine. For example, it makes up about 10% of the lipids of brain. It tends to be an abundant lipid in erythrocytes of most ruminant animals, where it replaces phosphatidylcholine entirely. In this instance, there is known to be a highly active phospholipase A that breaks down the glycerophospholipids, but not sphingomyelin. Like phosphatidylcholine, sphingomyelin tends to be most abundant in the plasma membrane, and especially in the outer leaflet, of cells.

The biosynthesis of sphingomyelin is distinct from that of phosphotidylcholine. Indeed, it involves transfer of phosphorylcholine from phosphatidylcholine to ceramide, liberating diacylglycerols, and catalyzed by a ceramide choline-phosphotransferase. The reaction takes place in the plasma membrane and Golgi, but much remains to be learned of the process.

In particular, the invention discloses phosphatidylcholine liquid formulations that have been fluidized with an oil composition containing a pre-determined ratio of omega 3 and omega 6 fatty acids. In order to make phosphatidylcholine supplements, either in capsules or liquid forms, oil must be added to the mix since pure phosphatides, especially those from soy lecithin, are solid at room temperature. The addition of oil is necessary to make the phosphatidylcholine composition fluid. The most important fatty acids in this regard are those over seventeen carbons in length, such as linoleic acid, gamma-linolenic acid, di-homo-gamma-linolenic acid, columbinic acid, eicosa-(n-6,9,13)-trienoic acid, arachidonic acid, timnodonic acid, hexaenoic acid, lignoceric acid, nervonic acid, or a combination thereof. These fatty acids may be included in the compositions of the present invention either individually or in mixtures with other fatty acids.

Essential Fatty Acids (EFAs) are long-chain polyunsaturated fatty acids derived from linolenic, linoleic, and some forms of oleic acids. EFAs are necessary fats that humans cannot synthesize, and must be obtained through diet. EFAs compete with undesirable fats (e.g., trans fats and cholesterol) for metabolism. Also, EFAs removes the HDL (High Density Lipoprotein) that is also considered beneficial for the body by capturing the undesirable LDL (Low Density Lipoprotein), and escort it to the liver where it is broken down and excreted.

There are two families of EFAs: omega 3 and omega 6. Omega 9 is necessary yet “non-essential” because the body can manufacture it in a modest amount, provided essential EFAs are present. The number following “omega” represents the position of the first double bond, counting from the terminal methyl group on the molecule. Linoleic Acid is an example of an omega 3 fatty acids, Linoleic Acid an example of an omega 6 fatty acids and Oleic Acid an example of an omega 9 fatty acid.

EFAs support the cardiovascular, reproductive, immune, and nervous systems. The human body needs EFAs to manufacture and repair cell membranes, enabling the cells to obtain optimum nutrition and expel harmful waste products. A primary function of EFAs is the production of prostaglandins, which regulate body functions such as heart rate, blood pressure, blood clotting, fertility, conception, and play a role in immune function by regulating inflammation and encouraging the body to fight infection. Essential Fatty Acids are also needed for proper growth in children, particularly for neural development and maturation of sensory systems, with male children having higher needs than females. Fetuses and breast-fed infants also require an adequate supply of EFAs through the mother's dietary intake. Because high heat destroys linolenic acid, cooking in linolenic-rich oils or eating cooked linolenic-rich fish is unlikely to provide a sufficient amount.

EFA deficiency is common in the United States, particularly Omega 3 deficiency due to the increased use of hydrogenated vegetable oil, and recently, over prescribing and consumption of Fish Oil. Essential fatty acid supplements include solutions comprising a mixture of omega 6 and omega 3 fatty acids, in ratio of from about 20/1, 10/1, 5/1, 4/1, 3/1, 2/1, 1/1, or less. It is intended herein that by recitation of such specified ranges, the ranges recited also include all those specific integer amounts between the recited ranges. For example, in the range of about 4/1, it is intended to also encompass 4.2/1, 3.8/1, 3.5/1, 3.2/1, 3/1, etc, without actually reciting each specific range therewith. Preferably the ratio between the omega 6 and omega 3 fatty acids is about 4/1 v/v.

Alpha Linolenic Acid (ALA) is the principal omega 3 fatty acid, which a healthy human will convert into eicosapentaenoic acid (EPA), and later into docosahexaenoic acid (DHA). Omega 3s are used in the formation of cell walls, making them supple and flexible, and improving circulation and oxygen uptake with proper red blood cell flexibility and function.

Omega 3 deficiencies are linked to decreased memory and mental abilities, tingling sensation of the nerves, poor vision, increased tendency to form blood clots, diminished immune function, increased triglycerides and “bad” cholesterol (LDL) levels, impaired membrane function, hypertension, irregular heart beat, learning disorders, menopausal discomfort, and growth retardation in infants, children, and pregnant women.

Food containing alpha linolenic acid includes flaxseed oil, flaxseed, flaxseed meal, hempseed oil, hempseed, walnuts, pumpkin seeds, Brazilian nuts, sesame seeds, avocados, some dark leafy green vegetables (e.g., kale, spinach, mustard greens, collards, etc.), canola oil (cold-pressed and unrefined), soybean oil, and others. Higher order omega 3 fatty acids (HUFA) include wild salmon, mackerel, sardines, anchovies, albacore tuna, cod liver oil, fish oil, and other cold water fish.

In one embodiment, alpha linolenic acid is cold pressed. For example, organic flaxseed oil is utilized with four parts of linoleic acid omega 6 oil as cold pressed in combination with organic sunflower oil as 4:1 omega 6/omega 3 ratio balanced oil. http:///www.mercurypoisoned.com/ Linoleic acid (LA) is the beginning of the omega 6 family. A healthy human with good nutrition can metabolize LA to gamma linolenic acid (GLA) and then to dihomo-gamma linolenic acid (DGLA), an eicosanoid, and then to arachidonic acid (AA), the lead eicosanoid. Both DGLA and AA along with the omega 3 eicosanoid EPA, form the important group of three eicosanoids that evolve into prostaglandins and leucotrienes, the hormone-like compounds which aid in many bodily functions including vital organ function and intracellular activity.

Some Omega 6s improve diabetic neuropathy, rheumatoid arthritis, PMS, skin disorders (e.g. psoriasis and eczema), inflammation, allergies, autoimmune conditions and aid in cancer treatment. Food containing linoleic acid includes safflower oil, sunflower seed, sunflower oil, hempseed oil, hempseed, pumpkin seeds, borage oil, evening primrose oil, black currant seed oil, among many others.

In one embodiment of the invention, a phosphatidylcholine composition is fluidized with the BodyBio Balance EFA 4:1 oil composition that contains linoleic acid and linolenic acid in the ratio of about 4:1. In another embodiment, 4 parts linoleic acid omega-6 oil is derived from a cold pressed, organic sunflower oil that is utilized along with I part of alpha linolenic acid derived from cold pressed, organic flaxseed oil in 4:1 omega 6/omega 3 ratio balanced oil.

The total amount of the essential oil added to the phosphatidylcholine product should be determined in view of the high concentration of already existing linoleic acid in the phosphatidylcholine concentrate products. In one embodiment, the final phosphatidylcholine product has about 10%, 15%, 20%, 30%, 40%, 50% or more of the EFA 4:1. In a preferred embodiment, the amount of the EFA 4:1 is about 20% of the final phosphatidylcholine product. In another preferred embodiment, the essential fatty acids are a blend of organic fatty acids that are cold pressed.

The elements of the combinations of the present invention work synergistically together because they have different, but complementary, mechanisms of action. For example, phosphatidylcholine functions to regulate protein and nucleic acid synthesis and regulation, perform hepatic detoxification, and provide structural support of cell membranes and membrane fluidity. Administration of a balanced ratio of essential fatty acids omega 6/omega 3 fatty acids such as EFAs 4:1 of the invention increases immune response and manufacture and repair of cell membranes enabling the cells to obtain optimum nutrition and expel harmful waste products. A primary function of EFAs is the production of prostaglandins, which regulate body functions such as heart rate, blood pressure, blood clotting, fertility, conception, and play a role in immune function by regulating inflammation and encouraging the body to fight infection. Essential Fatty Acids are also needed for proper growth in children, particularly for neural development and maturation of sensory systems.

Therefore, a composition combining phosphatidylcholine and essential fatty acids can be expected to function synergistically to increase structural support of cell membranes and membrane fluidity and simultaneously enhance the immune response enabling the cells to obtain optimum nutrition and expel harmful waste products. Therefore, the effects of combinations would be expected to be more than merely additive.

The fluidized phospholipid compositions of the invention are designed on the principle of “balanced nutrients”. The normal body keeps a healthy balance among essential nutrients that is a key in the well being and health of the individual. Unlike most therapies that cause an imbalance in the body of a sick individual who is already comprised by the sickness or the disease itself, the compositions and therapeutic methods of the present invention heal the subject individual by restoring the balance of essential nutrients to adjust it to a normal level in order to assist the body to fight the abnormal condition and/or ailments and to increase the ability of the immune system to fight the disease.

In one embodiment, the pharmanuetical composition of the invention additionally contains a stabilizer comprising a salt of the phytic acid, particularly an alkaline-earth salt and/or an alkaline salt of the phytic acid (e.g. calcium and or magnesium salts among others) as disclosed by U.S. Pat. No. 6,278,004 incorporated herein by reference in its entirety.

1. Pharmaneutical Compositions

The present invention provides pharmaneutical compositions comprising a therapeutically effective amount of phosphotidylcholine formulations that are fluidized with a pre-determined ratio of an oil composition containing a balanced ratio of omega 6 to omega 3 essential fatty acids.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In general, the combinations may be administered by the transdermal, intraperitoneal, intracranial, intracerebroventricular, intracerebral, intravaginal, intrauterine, oral, rectal, ophthalmic (including intravitreal or intracameral), nasal, topical (including buccal and sublingual), parenteral (including subcutaneous, intraperitoneal, intramuscular, intravenous, intradermal, intracranial, intratracheal, and epidural) administration.

The compositions of the invention may be administered as a single treatment, or repeated as enhancing or booster dosages, over a period up to and including one week to about 48 months or more.

The pharmaneutical compositions of the present invention, suitable for inoculation or for parenteral or oral administration, are in the form of sterile aqueous or non-aqueous solutions, suspensions, or emulsions, and can also contain auxiliary agents or excipients that are known in the art. Where necessary, the composition may also include a solubilizing agent and a local anesthetic such as lignocaine.

In addition, the compositions of the invention may be incorporated into biodegradable polymers allowing for sustained release of the compound, the polymers being implanted in the vicinity of where the delivery is desired, so that the composition is slowly released systemically.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The pharmaneutical composition formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Within other embodiments, the compositions may also be placed in any location such that the compounds or constituents are continuously released into the aqueous humor. The effective amount of the composition of the invention can be determined by standard clinical techniques on a case by case basis. In addition, in vitro assays may optionally be employed to help identify optimal dosage ranges.

In particular, the dosage of the compositions of the present invention will depend on a specific disease state and other clinical factors such as weight and condition of the human or animal and the route of administration of the compounds or compositions. The precise dose to be employed in the formulation, therefore, should be decided according to the judgment of the health care practitioner and each patient's circumstances. Effective doses may be extrapolated from dose-response curves derived from in vitro or animal model test systems.

Treating humans or animals between approximately 0.5 to 500 mg/kilogram is a typical broad range for administering the pharmaneutical composition of the invention. The methods of the present invention contemplate single as well as multiple administrations, given either simultaneously or over an extended period of time.

Preferred unit dosage formulations are those containing a daily dose or unit, daily sub-dose, or an appropriate fraction thereof, of the administered compositions. It should be understood that in addition to the compositions, particularly mentioned above, the formulations of the present invention may include other agents conventional in the art having regard to the type of formulation in question.

The pharmaneutical composition of the invention comprises a dry formulation, an aqueous solution, or both. Effective amounts of a phosphatidylcholine and the EFA 4:1 composition can each be formulated into the pharmaneutical composition for treatment, prevention or reduction of a wide variety of disease including CNS-related diseases or disorders. As used herein, a “pharmaneutical composition” includes compositions for human and veterinary use. Pharmaneutical compositions for parenteral (e.g., intravascular) administration are characterized as being sterile and pyrogen-free. One skilled in the art can readily prepare pharmaneutical compositions of the invention for enteral or parenteral use, for example by using the principles set forth in Remington's Pharmaceutical Science, 18^{th} edit. (Alphonso Gennaro, ed.), Mack Publishing Co., Easton, Pa., 1990.

Because phosphatidylcholine, linoleic acid and alpha linolenic acid are all soluble in oils or lipids, they can be conveniently formulated into a single pharmaneutical composition. Thus, in one embodiment, the invention provides a single-dose pharmaneutical composition comprising a phosphotidylcholine composition and an EFA 4:1 composition. Those constituents that are water soluble, such as for example, the liquid trace minerals, and electrolytes are generally not formulated into a single pharmaneutical composition with the phosphotidylcholine and EFAs compositions, but are rather formulated as separate compositions. However, the water soluble constituents, the phosphatidylcholine composition, and the EFA composition can be formulated into a single pharmaceutical composition as an emulsion, for example an oil-in-water emulsion or water-in-oil emulsion.

The pharmaneutical compositions of the invention can be in a form suitable for oral use, according to any technique suitable for the manufacture of oral pharmaceutical compositions as are within the skill in the art. For example, the phosphotidylcholine composition and the EFA composition can be formulated (either separately or together) into soft capsules, oily suspensions, or emulsions, optionally in admixture with pharmaceutically acceptable excipients. Suitable excipients for a phosphotidylcholine composition or EFA composition comprise oil-based media; e.g., archis oil, liquid paraffin, or vegetable oils such as olive oil.

In one embodiment, one or more oil compositions comprising linoleic acid and alpha linolenic acid in an approximately 4:1 (v/v) ratio are used to fluidized a phostidylcholine preparation. The term “EFA 4:1 composition” therefore refers to one or more compositions comprising linoleic acid and one or more compositions comprising alpha linolenic acid, which are mixed together and are used to fluidized a phosphatidylcholine preparation.

Any commercially available preparation comprising linoleic acid and alpha linolenic acid, or mixtures of the two in an approximately 4:1 (v/v) ratio, can be used as the EFA 4:1 composition in the present methods. Suitable EFA 4:1 compositions include the BodyBio Balance 4:1™ EFA oil available from BodyBio Inc. (Millville, N.J. USA), or any mixtures containing the essential fatty acids, such as for example, a mixture of cold pressed organic safflower or sunflower oil and flaxseed oil to yield a 4:1 ratio of linoleic acid to linolenic acid (4 parts Omega 6 to 1 part Omega 3).

The compositions of the invention are formulated into liquid or solid compositions, such as aqueous solutions, aqueous or oily suspensions, syrups or elixirs, emulsions, tablets, dispersible powders or granules, hard or soft capsules, optionally in admixture with pharmaceutically acceptable excipients.

2. Adjuvants, Carriers, and Diluents

As would be understood by one of ordinary skill in the art, when a composition of the present invention is provided to an individual, it can further comprise at least one of salts, buffers, adjuvants, or other substances which are desirable for improving the efficacy of the composition. Adjuvants are substances that can be used to specifically augment at least one immune response. Normally, the adjuvant and the composition are mixed prior to presentation to the immune system, or presented separately.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Adjuvants can be generally divided into several groups based upon their composition. These groups include lipid micelles, oil adjuvants, mineral salts (for example, AlK(SO₄)₂, AlNa (SO₄)₂, AlNH₄ (SO₄)), silica, kaolin, and certain natural substances, for example, wax D from Mycobacterium tuberculosis, substances found in Corynebacterium parvum, or Bordetella pertussis, Freund's adjuvant (DIFCO), alum adjuvant (Alhydrogel), MF-50 (Chiron) Novasomes™, or micelles, among others.

Suitable excipients for liquid formulation include water or saline, suspending agents such as sodium carboxymethylcellulose, methylcellulose, hydroxypropylmethyl-cellulose, sodium alginate, polyvinylpyrrolidone, gum tragacanth, and gum acacia; dispersing or wetting agents such as lecithin, condensation products of an alkylene oxide with fatty acids (e.g., polyoxethylene stearate), condensation products of ethylene oxide with long chain aliphatic alcohols (e.g., heptadecethyleneoxy-cetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyoxyethylene sorbitan monooleate).

Suitable excipients for solid formulations include calcium carbonate, sodium carbonate, lactose, calcium phosphate, or sodium phosphate; granulating and disintegrating agents such as maize starch, or alginic acid; binding agents such as starch, gelatin, or acacia; and lubricating agents such as magnesium stearate, stearic acids, or talc, and inert solid diluents such as calcium carbonate, calcium phosphate, or kaolin.

Other suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

Oral pharmaneutical compositions of the invention can contain one or more agents selected from the group consisting of sweetening agents, flavoring agents, coloring agents, and preserving agents in order to provide a pharmaneutically palatable preparation.

Liquid formulations according to the invention can contain one or more preservatives such as ethyl, n-propyl, or p-hydroxy benzoate; one or more coloring agents; one or more flavoring agents; or one or more sweetening agents such as sucrose, saccharin, or sodium or calcium cyclamate. Liquid pharmaneutical formulations according to the invention, especially those comprising a phosphotidylcholine composition fluidized with an EFA composition can additionally contain antioxidants such as tocopherol, sodium metabisulphite, butylated hydroxytoluene (BHT), butylated hydroxyanisole (BHA), ascorbic acid or sodium ascorbate. The pharmaneutical compositions of the invention are in the form of sterile, pyrogen-free preparations for example as a sterile aqueous solution, a suspension or an emulsion. Such pharmaneutical compositions can be formulated using the excipients described above for liquid formulations.

The compositions of the invention can be administered to the subject by any parenteral or enteral technique suitable for introducing the composition into the blood stream or gastrointestinal tract, including intravascular (e.g., intravenous and intraarterial) injection and oral administration. In a preferred embodiment, one or more compositions are administered to the subject both by mouth.

An “effective amount” of the compositions of the invention is any amount sufficient to therapeutically inhibit the progression of a disease or to prophylactically delay the onset of the disease symptoms. For example, the concentration of phosphatidylcholine in a composition can range from about 500 mg to about 50,000 mg or more, about 6000 mg to about 7500 mg, from about 2000 to about 5000 mg, and from about 3000 mg to about 4000, from about 10,000 mg to about 21000 mg phosphatidylcholine. It is intended herein that by recitation of such specified ranges, the ranges recited also include all those specific integer amounts between the recited ranges. For example, in the range of about 3000 mg to 4000 mg, it is intended to also encompass 3200 mg to 4300 mg, 3300 mg to 3800 mg, etc, without actually reciting each specific range therewith.

One of ordinary skill in the art can readily determine an appropriate temporal and interval regimen for administering the compositions of the invention. For example, the compositions of the invention can be administered once, twice or more daily, for one, two, three, four, five, six or seven days in a given a week. The length of time that the subject receives the composition can be determined by the subject's physician or other health care providers and caretakers, according to need.

In one embodiment of the invention, a phosphatidylcholine composition containing about 750 mg phosphatidylcholine is administered to a subject, for example two to three times daily, for consecutive or non-consecutive days in a given week. Another phosphotidylcholine composition which contains about 3600 mg to about 7200 mg phosphatidylcholine is administered, for example once or twice, to the same subject daily by mouth.

The invention also provides a phospholipid pharmaneutical pack or kit comprising one or more containers filled with one or more phospholipids compositions, and essential fatty acids in a pre-determined ratio for mixing with phospholipids compositions. In one embodiment, the kit comprises instructions for preparing and using the pharmaneutical composition and one or more of the following components: a phosphotidylcholine composition; 2) an EFA 4:1 composition; 3) an additive, such as flavors, colors, antibacterial agents, preservatives, or a combination thereof, and instruction for preparation and using the composition.

Generally, the ingredients are supplied either separately or mixed together in unit dosage form, for example, as a dry lyophilized powder or water free concentrate in a hermetically sealed container such as an ampoule or sachette indicating the quantity of one or more active agents. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

If a particular component is not included in the kit, the kit can optionally comprise information on where to obtain the missing component, for example an order form or uniform resource locator for the internet specifying a website where the component can be obtained.

The instructions provided with the kit describe the practice of the pharmaneutical composition of the invention as described above, and the route of administration and effective concentration and the dosing regimen for each of the compositions provided therein.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. All references discussed herein are incorporated by reference.

We claim:

1. A pharmaneutical composition comprising a phospholipid formulation that is fluidized with an essential oil composition containing a pre-determined ratio of omega 6 and omega 3 fatty acids and a suitable carrier.

2. The pharmaneutical composition of claim 1, wherein the phospholipid comprises phosphatidylserine, phosphatidylcholine, phosphatidylethanolamine, phosphatidylglycerol, phosphatidylinositol, phosphatidic acid, cardiolipin, sphingomyelin, ceramide, gangliosides, cerebrosides, or a combination thereof.

3. The pharmaneutical composition of claim 2, wherein the phospholipid comprises phosphotidylcholine.

4. The pharmaneutical composition of claim 2, wherein the phospholipids comprises phosphotidylcholine formulations having one or two acyl groups.

5. The pharmaneutical composition of claim 4, wherein the acyl groups have derived from myristic acid, palmitic acid, stearic acid, oleic acid, linoleic acid, linolenic acid, arachidic acid, arachidonic acid, or a combination thereof.

6. The pharmaneutical composition of claim 1, wherein the oil composition comprises a pre-determined ratio of linoleic acid and alpha linolenic acid.

7. The pharmaneutical composition of claim 6, wherein the linoleic acid and alpha linolenic acid are in a ratio of about 4:1.

8. The pharmaneutical composition of claim 4, wherein the phosphotidylcholine formulation comprise oral formulations.

9. The pharmaneutical composition of claim 6, wherein the oil composition is about 20% and the phosphotidylcholine is about 80% of total weight of the pharmaneutical composition.

10. The pharmaneutical composition of claim 1, wherein the omega 6 and omega 3 fatty acids are derived from a blend of organic fatty acids that are cold pressed.

11. A kit comprising a phospholipid formulation, an oil composition containing a predetermined ratio of omega 3 and omega 6 fatty acids, instructions for mixing the phospholipid formulation to obtain a fluidized phospholipid composition, and instructions to use the fluidized phospholipid composition.

12. The kit of claim 11, wherein the phospholipid comprises phosphatidylserine, phosphatidylcholine, phosphatidylethanolamine, phosphatidylglycerol, phosphatidylinositol, phosphatidic acid, cardiolipin, sphingomyelin, ceramide, gangliosides, cerebrosides, or a combination thereof.

13. The kit of claim 11, wherein the phosphotidylcholine comprises phosphotidylcholine formulations having one or two acyl groups.

14. The kit of claim 13, wherein the acyl groups have derived from myristic acid, palmitic acid, stearic acid, oleic acid, linoleic acid, linolenic acid, arachidic acid, arachidonic acid, or a combination thereof.

15. The kit of claim 11, wherein the oil composition comprises a pre-determined ratio of linoleic acid and alpha linolenic acid.

16. The kit of claim 15, wherein the linoleic acid and alpha linolenic acid are in a ratio of about 4:1.

17. The kit of claim 14, wherein the phosphotidylcholine formulation comprise intravenous and oral formulations.

18. The kit of claim 14, wherein the phosphotidylcholine formulation is a liquid or dry formulation.

19. The kit of claim 11 further comprising color, flavor, preservatives, antibacterial agents, or a combination thereof.
